(19) United States
(12) Patent Application Publication
(10) Pub. No.: US 2025/0195833 A1
(43) Pub. Date: Jun. 19, 2025

(54) VARIABLE RADIUS BRAID FOR A MEDICAL DEVICE

(71) Applicant: Boston Scientific Scimed, Inc., Maple Grove, MN (US)

(72) Inventors: Timothy Lawrence Rubesch, Blaine, MN (US); Gene Thomas Storbeck, Millis, MA (US); Anthony Frank Tassoni, JR., Andover, MN (US)

(21) Appl. No.: 18/972,663

(22) Filed: Dec. 6, 2024

Related U.S. Application Data
(60) Provisional application No. 63/609,709, filed on Dec. 13, 2023.

(51) Int. Cl. A61M 25/00; A61B 17/12

(52) U.S. Cl. CPC A61M 25/0053; A61B 17/12172

(57) ABSTRACT

A tubular medical device includes a flexible body having a proximal portion and a distal portion including a distal end. The flexible body is formed of a plurality of braided filaments. The flexible body includes a longitudinal axis extending from the proximal portion to the distal end. The flexible body includes a first braid density at a first radial location and a second braid density at a second radial location, the first radial location and the second radial location being at a same axial location along the longitudinal axis.

### CROSS REFERENCE TO RELATED APPLICATIONS

This application claims priority to U.S. Provisional Patent Application No. 63/609,709 entitled “VARIABLE RADIUS BRAID FOR A MEDICAL DEVICE,” filed Dec. 13, 2023, which is hereby incorporated by reference in its entirety.

### TECHNICAL FIELD

The present disclosure relates to medical devices for use in a patient. More specifically, the present disclosure relates to braided tubular medical devices for use in patients and methods of manufacturing the same.

### BACKGROUND

Various braided medical devices are used for treating conditions in a patient or delivering materials or devices into a patient. When delivering or implanting such devices into the patient's body it is critical that the braided device has sufficient properties, such as steerability, pushability, and torque transmission to ensure delivery. The ease of operation by which the medical device can be delivered is crucial from several aspects such as requirements to comply with time limits for quick treatment or overall safe and accurate positioning or maneuvering of the device at the target site.

With standard braid designs, such as those having constant pic per inch (PPI) or simple axial pic change, it can be challenging to find the right balance of shaft mechanical performance. When designing a shaft, the braid pattern typically influences one or potentially two mechanical properties (1-Kink, 2-Torque, 3-Push, and 4-Flexibility) towards overall product performance. What is needed is a braid pattern that can increase performance in more than two mechanical properties.

### SUMMARY

Example 1 is a tubular medical device. The tubular medical device includes a flexible body having a proximal portion and a distal portion including a distal end. The flexible body is formed of a plurality of braided filaments. The flexible body includes a longitudinal axis extending from the proximal portion to the distal end. The flexible body includes a first braid density at a first radial location and a second braid density at a second radial location, the first radial location and the second radial location being at a same axial location along the longitudinal axis.

Example 2 is the medical device of Example 1, further comprising one or more polymeric layers configured to encase the flexible body.

Example 3 is the medical device of any of Examples 1 or 2, wherein the same axial location is within the distal portion.

Example 4 is the medical device of any of Examples 1-3, wherein the distal portion curves in an unconstrained configuration.

Example 5 is the medical device of Example 4, wherein the distal portion curves in a direction towards a high braid density.

Example 6 is the medical device of any of Examples 1-5, wherein the flexible body has a third braid density at a third radial location, the third radial location being at the same axial location along the longitudinal axis.

Example 7 is the medical device of c Example 6, wherein the flexible body has a fourth braid density at a fourth radial location, the fourth radial location being at the same axial location along the longitudinal axis.

Example 8 is the medical device of any of Examples 1-7, wherein the tubular medical device is an implantable device.

Example 9 is the medical device of Example 8, wherein the tubular medical device is an occlusion device.

Example 10 is the medical device of any of Examples 1-7, wherein the tubular medical device is an introduction device.

Example 11 is the medical device of Example 10, wherein the tubular medical device is a catheter.

Example 12 is the medical device of any of Examples 1-11, wherein the flexible body includes one or more reinforcing regions.

Example 13 is the medical device of any of Examples 1-12, wherein at least one of the plurality of braided filaments is radiopaque.

Example 14 is the medical device of any of Examples 1-13, wherein the plurality of braided filaments have a cross-section that is rectangular, oval, circular, dome shaped, or polygonal.

Example 15 is the medical device of any of Examples 1-14, wherein the flexible body has a third braid density at a third radial location, the third radial location being spaced from the same axial location along the longitudinal axis.

Example 16 is a tubular medical device. The tubular medical device includes a flexible body having a proximal portion and a distal portion including a distal end. The flexible body is formed of a plurality of braided filaments. The flexible body includes a longitudinal axis extending from the proximal portion to the distal end. The flexible body has a first braid density at a first radial location and a second braid density at a second radial location. The first braid density is greater than the second braid density, and the first radial location and the second radial location are at a same axial location along the longitudinal axis.

Example 17 is the medical device of Example 16, further comprising one or more polymeric layers configured to encase the flexible body.

Example 18 is the medical device of Example 16, wherein the distal portion curves in an unconstrained configuration.

Example 19 is the medical device of Example 18, wherein an introduction sheath or stylet constrains the distal portion.

Example 20 is the medical device of Example 18, wherein the distal portion curves in a direction towards a high braid density.

Example 21 is the medical device of Example 16, wherein the flexible body has a third braid density at a third radial location, the third radial location being at the same axial location along the longitudinal axis.

Example 22 is the medical device of Example 21, wherein the flexible body has a fourth braid density at a fourth radial location, the fourth radial location being at the same axial location along the longitudinal axis.

Example 23 is the medical device of claim 16, wherein the tubular medical device is an implantable device.

Example 24 is the medical device of Example 23, wherein the tubular medical device is an occlusion device.

Example 25 is the medical device of Example 16, wherein the tubular medical device is an introduction device.

Example 26 is the medical device of Example 25, wherein the tubular medical device is a catheter.

Example 27 is the medical device of Example 16, wherein the flexible body includes one or more reinforcing regions.

Example 28 is the medical device of Example 16, wherein at least one of the plurality of braided filaments is radiopaque.

Example 29 the medical device of Example 16, wherein the plurality of braided filaments have a cross-section that is rectangular, oval, circular, dome shaped, or polygonal.

Example 30 is the medical device of Example 16, wherein the flexible body has a third braid density at a third radial location, the third radial location being spaced from the same axial location along the longitudinal axis.

Example 31 is a tubular medical device. The tubular medical device includes a flexible body having a proximal end and a distal portion including a distal end. The flexible body is formed of a plurality of braided filaments. The flexible body includes a longitudinal axis extending from the proximal portion to the distal end. One or more polymeric layers is configured to encase the flexible body. The flexible body has a first braid density at a first radial location, a second braid density at a second radial location, and a third braid density at a third radial location. The first radial location and the second radial location are at a same axial location along the longitudinal axis.

Example 32 is the medical device of Example 31, wherein the third radial location is at the same axial location.

Example 33 is the medical device of Example 31, wherein the third radial location is spaced from the same axial location along the longitudinal axis.

Example 34 is the medical device of Example 31, wherein the same axial location is within the distal portion.

Example 35 is a tubular medical device. The tubular device includes a flexible body having a proximal portion and a distal portion including a distal end. The flexible body is formed of a plurality of braided filaments. The flexible body includes a longitudinal axis extending from the proximal portion to the distal end. One or more polymeric layers is configured to encase the flexible body. The flexible body has a first braid density at a first radial location, a second braid density at a second radial location, a third braid density at a third radial location, and a fourth braid density at a fourth radial location. The first radial location and the second radial location are at a same axial location along the longitudinal axis.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a perspective view of a braided tubular medical device, in accordance with the disclosure.

FIG. 2A is an illustration of a braided portion of a device having a radially uniform braid density, in accordance with the disclosure.

FIG. 2B is an illustration of a braided portion of a device having a radially variable braid density, in accordance with the disclosure.

FIG. 3 is a perspective view of the braided tubular medical device of FIG. 1 in an unconstrained state, in accordance with the disclosure.

FIG. 4 is a perspective view of the braided tubular medical device of FIG. 1 in an unconstrained state, in accordance with the disclosure.

FIG. 5 is a perspective view of a braided tubular medical device, in accordance with the disclosure.

FIG. 6 is a perspective view of a braided tubular medical device, in accordance with the disclosure.

FIG. 7A is a cross-section of a braided tubular medical device, in accordance with the disclosure.

FIG. 7B illustrates a first side of the braided tubular medical device of FIG. 7A, in accordance with the disclosure.

FIG. 7C illustrates a second side of the braided tubular medical device of FIG. 7A, in accordance with the disclosure.

FIG. 8A is a cross-section of a braided tubular medical device, in accordance with the disclosure.

FIG. 8B illustrates a first and second portion of the braided tubular medical device of FIG. 8A, in accordance with the disclosure.

FIG. 8C illustrates a third and fourth portion of the braided tubular medical device of FIG. 8A, in accordance with the disclosure.

FIGS. 9A-9E illustrate various cross-sections for filaments forming a braided tubular medical device, in accordance with the disclosure.

FIG. 10 is a cross-section of a braided tubular medical device, in accordance with the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

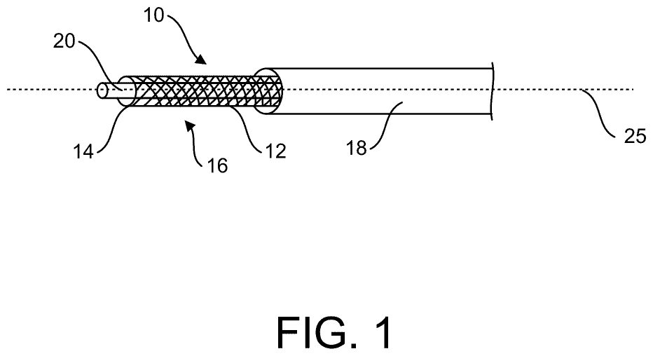

FIG. 1 is a perspective view of a braided tubular medical device 10, in accordance with the disclosure. Braided tubular medical device 10 can include an implantable device configured to treat a portion of patient, or it can form part of an introduction device to deliver medicaments or other devices to a patient. For example, the braided tubular medical device 10 can be an occlusion device, stent, dilator, filter, anchor, sheath, or a catheter. Braided tubular medical device 10 includes a flexible body 12 formed of a plurality of filaments braided together. In one embodiment, the plurality of filaments are formed of a single type of material. In another embodiment, the plurality of filaments are formed of a plurality of materials.

In some embodiments, the flexible body 12 acts as a reinforcement for a tubular device, such as a catheter or sheath, to help increase the rigidity, torqueability, or steerability of the tubular device. In some embodiments, the flexible body 12 is placed over or the outside of a polymeric tubular structure. In some embodiments, the flexible body 12 is sandwiched between multiple polymeric layers or embedded into a polymer to incorporate the flexible body 12 into the tubular device. In some embodiments, the flexible body 12 includes no covering, coating, or additional material surrounding the flexible body 12.

The plurality of filaments may be any material suitable for implanting in a human or animal body. For example, stainless steel, tungsten, and nitinol, may be used as a material for the plurality of filaments. However, suitable materials for embodiments of the braiding are various and include shape memory materials, metal, superelastic alloys, and polymers.

The flexible body 12 includes a distal end 14 and a proximal end (not shown) opposite the distal end 14. The flexible body 12 includes a distal portion 16 that extends from the distal end 14 proximally to a proximal portion (not shown). The distal portion 16 of the flexible body 12 has a natural curvature in an unconstrained configuration. In order to deliver the braided tubular medical device 10 to a desired location in a patient, the braided tubular medical device 10 may be constrained externally by an introduction sheath or catheter 18 or internally by a stylet or mandrel 20. While both an introduction sheath or catheter 18 and a stylet or mandrel 20 are illustrated, it is understood that the braided tubular medical device 10 can be constrained by only one of the two.

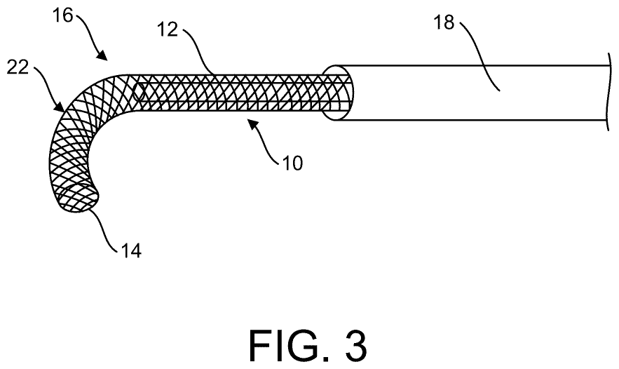

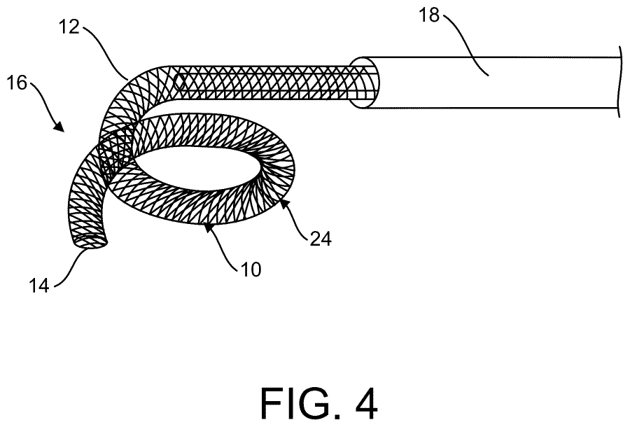

As illustrated in FIGS. 3 and 4, the distal portion 16 of the flexible body 12 is able to obtain the natural curvature when the flexible body 12 is free from the constraints of the introduction sheath or catheter 16 and stylet or mandrel 18. As illustrated, the amount of flexible body 12 that is free from a constraint will determine the amount of curvature in the distal portion 16. In FIG. 3, the braided tubular medical device 10 begins to form a single curve 22 in the distal portion 16. The single curve 22 allows the distal end 14 to be offset from a longitudinal axis 25 that passes through the braided tubular medical device 10 in a constrained configuration. The single curve 22 can be beneficial for introducing the braided tubular medical device 10 into a patient, for example during navigating the tortuous passageways of the vascular system.

In FIG. 4, the braided tubular medical device 10 is extended far enough to form a spiral 24 in the distal portion 16. The spiral 24 can be used to anchor the braided tubular medical device 10 in a desired location in a patient.

The natural curvature in the distal portion 16 of the flexible body 12 is created by radially varying the braid density, also known as Pics Per Inch (PPI), around the circumference of the braided tubular medical device 10. This variation is limited to the distal portion 16, or to any region desired to achieve a natural curve when unconstrained. The braid density is greater at a location of the circumference under the curve in the distal portion 16. A location of the opposite side of the circumference, i.e. the top of the curve, has a smaller braid density then the portion of the circumference below the curve.

PPI is based on wire volume, core placement, and speed of material passing through a braider. To create radial variation in the braid density or PPI, the core of a material is allowed to be placed in different locations of the braider from center of the machine. This new location not only augments the braid design, but delivers new pic counts in Radial directions around the circumference of the braided tubular medical device 10. This allows for the braid configuration to change on different sides of the flexible body 12 during manufacture.

Radially varying the braid density allows for greater control of the flexible body 12. By placing a high PPI that allows for bend and flex in ¼ or ½ of the diameter of the flexible body 12 in a linear direction and low PPI that allows for stiffness and
push in another ¼ or ½ of the diameter of the flexible body 12 in a radial direction, the flexible body 12 is able to obtain a natural curvature without any additional down stream processing, such as heat treatment. In general, a low PPI equates to more pushability and torque transmission along a body of a medical device, and a high PPI equates to increased flexibility along the body.

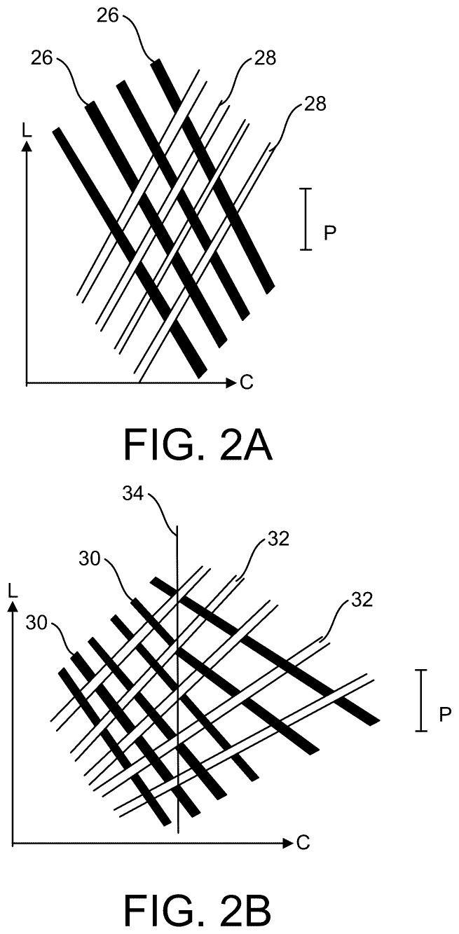

FIG. 2A is an illustration of a braided portion of a device having a radially uniform braid density, in accordance with the disclosure. As illustrated in FIG. 2A, the braid is formed of a plurality of filaments 26, 28. The plurality of filaments 26, 28 are shown in different colors for ease of presentation. In some embodiments, the plurality of filaments 26, 28 may be formed of a single material. In some embodiments, the plurality of filaments 26, 28 may be formed of a plurality of materials. In some embodiments, the plurality of filaments 26, 28 may have the same size and cross-sectional shape. In some embodiments, the plurality of filaments 26, 28 may have different sizes and cross-sectional shapes.

The plurality of filaments 26, 28 are braided together to form a radially uniform braid density around the circumference of a flexible body. In FIG. 2A, the braided portion is illustrated as flat. The circumference of the flexible body is illustrated in the C direction, while the longitudinal axis of the body is illustrated in the L direction. As can be seen in FIG. 2A, the pics P of the braided section are uniform around the circumference C. The proximal portion of a tubular braided medical device, or any portion of the device that is not desired to have a natural curve, can include a radially uniform braid density.

FIG. 2B is an illustration of a braided portion of a device having a radially variable braid density, in accordance with the disclosure. As illustrated in FIG. 2B, the braid is formed of a plurality of filaments 30, 32. The plurality of filaments 30, 32 are shown in different colors for ease of presentation. In some embodiments, the plurality of filaments 30, 32 may be formed of a single material. In some embodiments, the plurality of filaments 30, 32 may be formed of a plurality of materials. In some embodiments, the plurality of filaments 30, 32 may have the same size and cross-sectional shape. In some embodiments, the plurality of filaments 30, 32 may have different sizes and cross-sectional shapes.

The plurality of filaments 30, 32 are braided together to form a radially variable braid density around the circumference of a flexible body. In FIG. 2B, the braided portion is illustrated as flat. The circumference of the flexible body is illustrated in the C direction, while the longitudinal axis of the body is illustrated in the L direction. As can be seen in FIG. 2B, the pics P of the braided section are variable around the circumference C. In FIG. 2B, the right side of line 34 illustrates a top half of the circumference of a flexible body having a lower braid density and the left side of line 34 illustrates a bottom half of the circumference of a flexible body have a higher braid density. In this configuration, a curve would form in the flexible tubular body that curves to the left side of line 34, along the second of higher braid density.

While the braid in FIGS. 2A and 2B are illustrated as having single filaments braided together, it is understood that other arrangements of filaments can be used to create a natural curve in a flexible body, as long as there is variation radially in the braid density. For example, the braid can be formed of groups of two filaments that are braided together, where two filaments are adjacent one another, then side by side alternately pass under two filaments, then over two filaments, and so on. Additionally, the braid can include additional filaments to modify properties of the flexible body 12. For example, the flexible body 12 can include one or more radiopaque filaments.

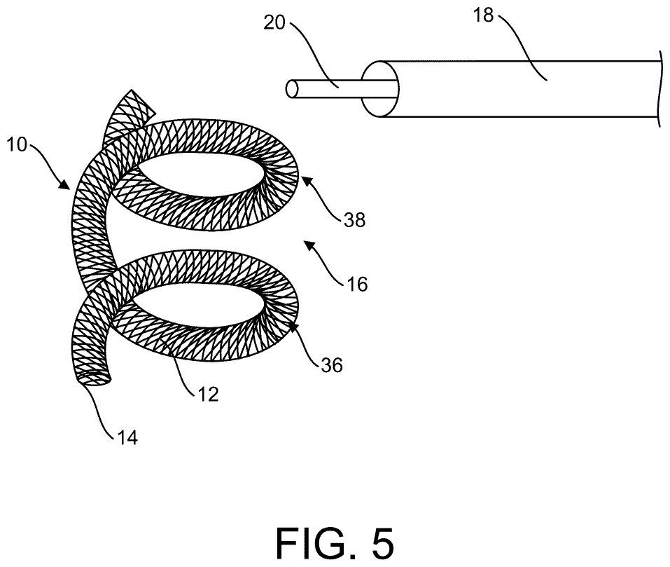

FIG. 5 is a perspective view of a braided tubular medical device 10, in accordance with the disclosure. In FIG. 5, the braided tubular medical device 10 can be configured as an occluding device such as, for example, the Boston Scientific Corp. Embold™ product, embolic coils, marker, filter, anchor, or other device configured to be implanted in a patient. The distal portion 16 of the braided tubular medical device 10 obtains a spiral shape when in an unconstrained configuration. The spiral includes a first curved portion 36 and a second curved portion 38. The first curved portion 36 and the second curved portion 38 have the same curvature. The braided tubular medical device 10 can have a length such that it can be placed at a desired location, and the introduction sheath or catheter 18 and/or the stylet or mandrel 20 can be withdrawn, leaving the entire device 10 behind in an unconstrained configuration.

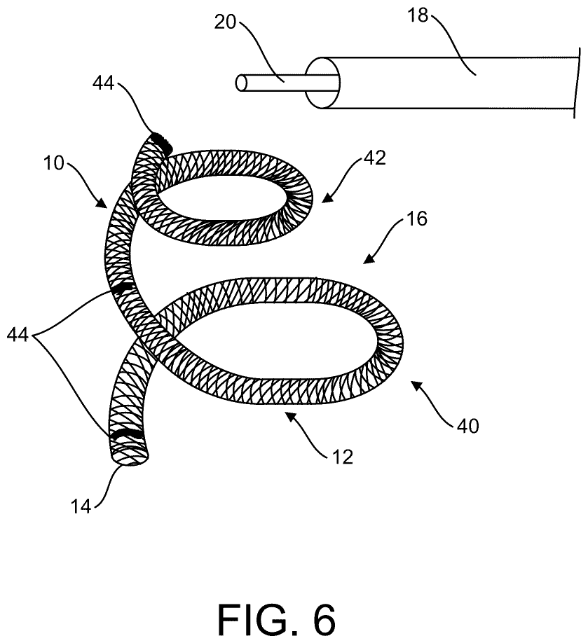

FIG. 6 is a perspective view of a braided tubular medical device having regions of variable curvature, in accordance with the disclosure. In FIG. 6, the braided tubular medical device 10 can be configured as an occluding device, marker, filter, anchor, or other device configured to be implanted in a patient. The distal portion 16 of the braided tubular medical device 10 obtains a spiral shape when in an unconstrained configuration. The spiral includes a first curved portion 40 and a second curved portion 42. The first curved portion 40 and the second curved portion 42 have different curvatures. This is achieved by radially varying the braid density along two different axial sections of the distal portion 16. When the braided tubular medical device 10 is at a desired location the introduction sheath or catheter 18 and/or the stylet or mandrel 20 can be withdrawn, leaving the entire device 10 behind. The braided tubular medical device 10 can include reinforcement regions or markers 44. In some embodiments, the reinforcement regions or markers 44 can include a polymer doped with radiopaque material, or a radiopaque metal. The reinforcement regions or markers 44 can be positioned along the device 10 to help identify the distal and proximal ends of the device 10, as well as intermediate portions of the device 10, during implantation.

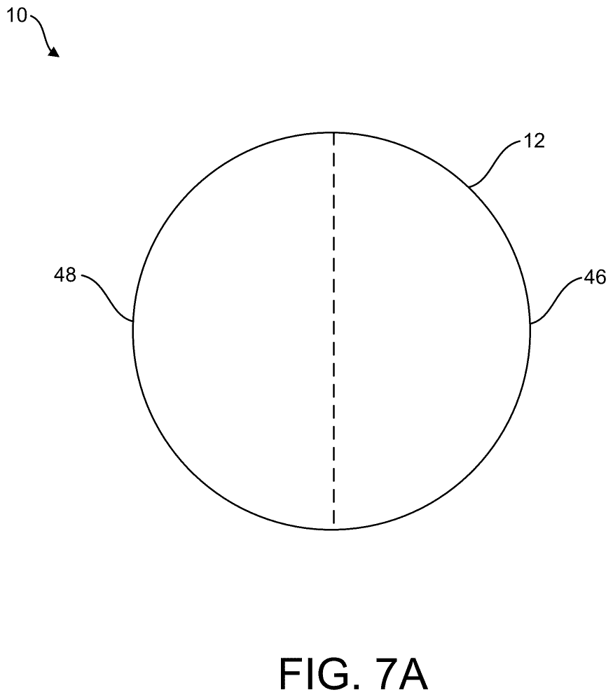

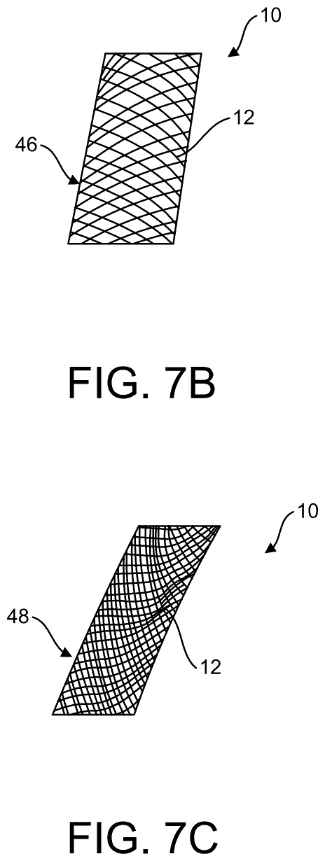

FIG. 7A is a cross-section delineating regions of varying braid density of a braided tubular medical device 10, in accordance with the disclosure. FIG. 7A illustrates a first half 46 of a flexible body 12 having a braid density of 30 PPI and a second half 48 of the flexible body 12 having a braid density of 70 PPI. Such an arrangement would lead to a curve in the direction of the second half when the flexible body 12 is in an unconstrained state.

FIG. 7B illustrates the first half 46 of the braided tubular medical device 10 of FIG. 7A, in accordance with the disclosure. FIG. 7C illustrates the second half 48 of the braided tubular medical device 10 of FIG. 7A, in accordance with the disclosure. As can be seen in FIGS. 7B and 7C, the flexible body 12 has a higher braid density on the second half 48 than the first half 46 of flexible body 12.

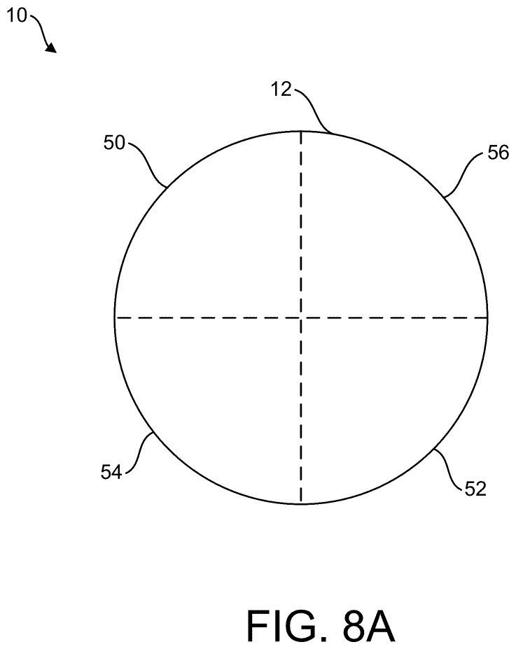

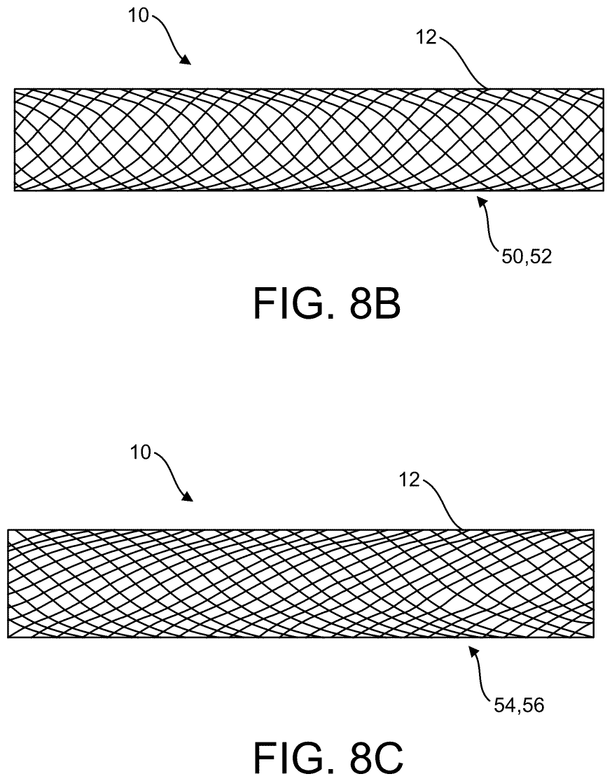

FIG. 8A is a cross-section delineating regions of varying braid density of a braided tubular medical device 10, in accordance with the disclosure. In FIG. 8A, the braided tubular medical device 10 includes a radially variable braid density in four sections of the circumference of the flexible body 12. A first section 50 and a second section 52 of the flexible body 12 have a braid density of 30 PPI. A third section 54 and a fourth second 56 of the flexible body 12 has a braid density of 60 PPI. The first section 50, second section 52, third section 54, and fourth section 56 of the flexible body each include ¼ of the circumference of the flexible body 12. As illustrated, the first section 50 is opposite the second section 52 and the third section 54 is opposite the fourth section 56. By having four sections of radially variable braid density, the flexible body 12 can be further optimized, and overall performance can be increased. In some embodiments, each of first section 50, second section 52, third section 54, and fourth section 56 have braid densities that are different. The first section 50 and the second section 52 provide increased tensile strength while the third section 54 and the fourth section 56 provide kink resistance to navigate tortuous anatomies with reduced trace force. This design reduces elongation of the flexible body 12 using just the braid design without the added complexity of needing to incorporate longitudinal reinforcement.

FIG. 8B illustrates the first section 50 and the second section 52 of the braided tubular medical device of FIG. 8A, in accordance with the disclosure. FIG. 8C illustrates the third section 54 and the fourth section 56 of the braided tubular medical device of FIG. 8A, in accordance with the disclosure. As can be seen in FIGS. 8B and 8C, the flexible body 12 has a higher braid density in the third section 54 and the fourth section 5 than in the first section 50 and second section 52 illustrated in FIG. 8B.

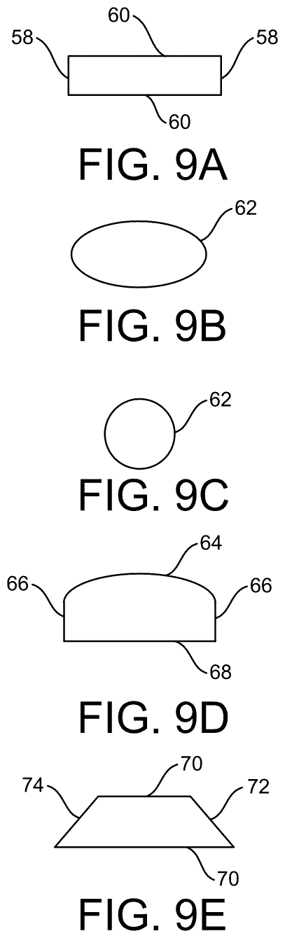

FIGS. 9A-9E illustrate various cross-sectional arrangements for the filaments forming the braided tubular medical device 10, in accordance with the disclosure. The filaments forming the flexible body 12 can include a variety of cross-sections and can include a thickness that is less than the width. In various embodiments, the filaments have a thickness sufficient to provide structural support to the flexible body, while maintaining substantial flexibility. FIG. 9A illustrates a filament having a rectangular cross-section. The rectangular cross-section includes a first pair of surfaces 58 that are orthogonal to a second pair of surfaces 60. FIG. 9B illustrates a filament having an oval cross-section. The oval cross-section includes a single surface 62. FIG. 9C illustrates a filament having a circular cross-section. Like the oval cross-section, the circular cross-section includes a single surface 62. FIG. 9D illustrates a filament having a dome shaped cross-section. The dome shaped cross-section includes a curved surface 64, and a first pair of parallel surfaces 66 that are orthogonal to a flat surface 68 opposite of the curved surface 64. FIG. 9E illustrates a filament having a polygonal cross-section. The polygonal cross-section includes a pair of parallel surfaces 70 that are intersected by a first angled surface 72 and a second angled surface 74.

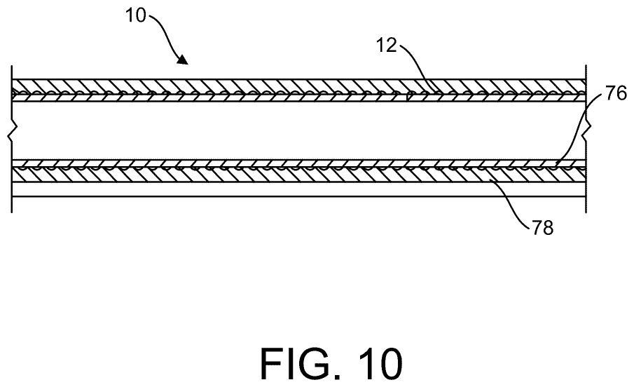

FIG. 10 is a cross-section of a braided tubular medical device 10, in accordance with the disclosure. FIG. 10 shows the flexible body 12 of the braided tubular medical device 10 being encased in one or more polymeric sleeve. The flexible body 12 is encased by an inner polymeric layer 76 and an outer polymeric layer 78. In some embodiments, the inner polymeric layer 76 and the outer polymeric layer 78 can be formed of the same material. In some embodiments, the inner polymeric layer 76 and the outer polymeric layer 78 are different materials. The inner polymeric layer 76 and the outer polymeric layer 78 can be configured to enclose the flexible body 12 so that fluids may pass through the braided tubular medical device 10. The embodiment of FIG. 10 can form a portion of a catheter, sheath, dilator, or other tubular structure configured for introduction into a patient. The portion can be a distal portion that is configured to have a preset curve in an unconstrained configuration, to allow for navigation though tortuous passageways within a patient.

In one embodiment, the inner polymeric layer 76 is formed of extruded Polytetrafluoroethylene (PTFE) and the outer polymeric layer 78 is formed of Polyether Block Amide, for example PEBAX 4033 SA01 which is a thermoplastic elastomer made of flexible polyether and rigid polyamide. The flexible body 12 can include a radially variable braid density as discussed above. The braid density for various portions of the flexible body 12 can range from 15 PPI to 90 PPI and can include a braid angle in the range of 10 to 50. The surface area coverage for the flexible body 12 can include a range of approximately 10% to 40%.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A tubular medical device, the tubular medical device comprising:
a flexible body having a proximal portion and a distal portion including a distal end;
the flexible body being formed of a plurality of braided filaments;
the flexible body having a longitudinal axis extending from the proximal portion to the distal end;
wherein, the flexible body has a first braid density at a first radial location and a second braid density at a second radial location, the first braid density being greater than the second braid density, and the first radial location and the second radial location being at a same axial location along the longitudinal axis.

2. The medical device of claim 1, further comprising one or more polymeric layers configured to encase the flexible body.

3. The medical device of claim 1, wherein the distal portion curves in an unconstrained configuration.

4. The medical device of claim 3, wherein an introduction sheath or stylet constrains the distal portion.

5. The medical device of claim 3, wherein the distal portion curves in a direction towards a high braid density.

6. The medical device of claim 1, wherein the flexible body has a third braid density at a third radial location, the third radial location being at the same axial location along the longitudinal axis.

7. The medical device of claim 6, wherein the flexible body has a fourth braid density at a fourth radial location, the fourth radial location being at the same axial location along the longitudinal axis.

8. The medical device of claim 1, wherein the tubular medical device is an implantable device.

9. The medical device of claim 8, wherein the tubular medical device is an occlusion device.

10. The medical device of claim 1, wherein the tubular medical device is an introduction device.

11. The medical device of claim 10, wherein the tubular medical device is a catheter.

12. The medical device of claim 1, wherein the flexible body includes one or more reinforcing regions.

13. The medical device of claim 1, wherein at least one of the plurality of braided filaments is radiopaque.

14. The medical device of claim 1, wherein the plurality of braided filaments have a cross-section that is rectangular, oval, circular, dome shaped, or polygonal.

15. The medical device of claim 1, wherein the flexible body has a third braid density at a third radial location, the third radial location being spaced from the same axial location along the longitudinal axis.

16. A tubular medical device, the tubular medical device comprising:
a flexible body having a proximal end and a distal portion including a distal end;
the flexible body being formed of a plurality of braided filaments;
the flexible body having a longitudinal axis extending from the proximal portion to the distal end; and
one or more polymeric layers configured to encase the flexible body;
wherein, the flexible body has a first braid density at a first radial location, a second braid density at a second radial location, a third braid density at a third radial location, and wherein the first radial location and the second radial location are at a same axial location along the longitudinal axis.

17. The medical device of claim 1, wherein the third radial location is at the same axial location.

18. The medical device of claim 1, wherein the third radial location is spaced from the same axial location along the longitudinal axis.

19. The medical device of claim 1, wherein the same axial location is within the distal portion.

20. A tubular medical device, the tubular medical device comprising:
a flexible body having a proximal portion and a distal portion including a distal end;
the flexible body being formed of a plurality of braided filaments;
the flexible body having a longitudinal axis extending from the proximal portion to the distal end;
one or more polymeric layers configured to encase the flexible body;
wherein, the flexible body has a first braid density at a first radial location, a second braid density at a second radial location, a third braid density at a third radial location, a fourth braid density at a fourth radial location, and wherein the first radial location and the second radial location are at a same axial location along the longitudinal axis.
